(19) United States
(12) Patent Application Publication
(10) Pub. No.: US 2020/0063181 A1
(43) Pub. Date: Feb. 27, 2020

(54) FLUORESCENT PROBE

(71) Applicant: NATIONAL SUN YAT-SEN UNIVERSITY, KAOHSIUNG CITY (TW)

(72) Inventors: SHU-CHEN HSIEH, Kaohsiung City (TW); PEI-YING LIN, Kaohsiung City (TW)

(21) Appl. No.: 16/665,423

(22) Filed: Oct. 28, 2019

Related U.S. Application Data
(63) Continuation-in-part of application No. 15/622,592, filed on Jun. 14, 2017.

(30) Foreign Application Priority Data
May 9, 2017 (TW) 106115335

(51) Int. Cl. C12Q 1/26; C12N 11/14; G01N 21/64

(52) U.S. Cl. CPC C12Q 1/26; C12N 11/14; G01N 2021/6432; G01N 21/6489; C12Y 101/03004

(57) ABSTRACT

A fluorescent probe is obtained via hydrolysis and condensation reaction using 3-glycidoxypropyl trimethoxysilane. The fluorescent probe includes a silicon oxide core and a self-assembled monolayer. The self-assembled monolayer has an epoxide group, and joins the silicone oxide core by a covalent bond. The epoxide group of the fluorescent probe can form a conjugated bond with a molecule with an amino group via an aminolysis reaction, forming a nanoparticle including the molecule and the fluorescent probe.

### CROSS REFERENCE TO RELATED APPLICATIONS

This is a continuation-in-part application of U.S. patent application Ser. No. 15/622,592 filed on Jun. 14, 2017, and claims the benefit of Taiwan application serial No. 106115335, filed on May 9, 2017, the entire contents of which are incorporated herein by reference.

### BACKGROUND OF THE INVENTION

### 1. Field of the Invention

The present invention relates to a probe and, more particularly, to a fluorescent probe able to emit fluorescence.

### 2. Description of the Related Art

Quantum dots (QD) are semiconductor particles with only several nanometers in size. Based on their excellent optical properties, QDs are usually adapted to fluorescent probes for biological analysis. As an example, CdSe/ZnS QDs, assembled with glucose oxidase (GOx), can be used to monitor glucose concentration of a glucose solution based on their fluorescence quenching by the H₂O₂ molecules produced by GOx.

However, GOx easily lose their enzymatic activity if GOx are immobilized on the CdSe/ZnS QDs. As a result, sensitivity and specificity of the CdSe/ZnS QDs on detecting glucose concentration decrease. In light of this, it is necessary to develop a fluorescent probe on which GOx with enzymatic activity can be immobilized.

### SUMMARY OF THE INVENTION

It is therefore an objective of the present invention to provide a fluorescent probe on which glucose oxidase with enzymatic activity can be immobilized.

In an aspect, a fluorescent probe is obtained via hydrolysis and condensation reaction using 3-glycidoxypropyl trimethoxysilane. The fluorescent probe includes a silicon oxide core and a self-assembled monolayer. The self-assembled monolayer has an epoxide group, and joins the silicone oxide core by a covalent bond. The fluorescent probe can emit fluorescence, and the epoxide group of the fluorescent probe can form a conjugated bond with a molecule with an amino group (such as various amino acids and proteins) via an aminolysis reaction. Thus, the molecule can be stably immobilized on the self-assembled monolayer of the fluorescent probe. Moreover, the maximum fluorescence emission (λₑₘ) of the fluorescent probe changes due to the immobilization of the molecule. Thus, the maximum fluorescence emission (λₑₘ) of the fluorescent probe can be used to determine whether the molecule able to be immobilized on is present in a solution. In addition, in the situation that the molecule is glucose oxidase, due to the fluorescent probe has a pH value about 7, glucose oxidase immobilized on the fluorescent probe can still show enzymatic activity.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a schematic diagram illustrating of the fluorescent probe according to an embodiment of the present invention.

FIG. 2a is an AFM particle size distribution histogram of the fluorescent probe according to the embodiment of the present invention.

FIG. 2b is a TEM image of the fluorescent probe according to the embodiment of the present invention.

FIG. 3 is a SERS spectrum of the fluorescent probe according to the embodiment of the present invention.

FIG. 4a is a PL spectrum of the fluorescent probe (group B0) according to the embodiment of the present invention.

FIG. 4b is a PL spectrum of the fluorescent probe (group B1) according to the embodiment of the present invention after the fluorescent probe is added to a solution including glucose oxidase.

FIG. 4c is a PL spectrum of the fluorescent probe (group B2) according to the embodiment of the present invention after the fluorescent probe is added to a solution including cisplatin.

FIG. 4d is a PL spectrum of the fluorescent probe (group B3) according to the embodiment of the present invention after the fluorescent probe is added to a solution including arginine.

FIG. 4e is a PL spectrum of the fluorescent probe (group B4) according to the embodiment of the present invention after the fluorescent probe is added to a solution including octadecylamine.

FIG. 4f is a PL spectrum of the fluorescent probe (group B5) according to the embodiment of the present invention after the fluorescent probe is added to a solution including octadecane.

FIG. 5 is a PL spectrum illustrating the fluorescent intensity of the fluorescent probe on which glucose oxidase is immobilized after glucose treatment.

### DETAILED DESCRIPTION OF THE INVENTION

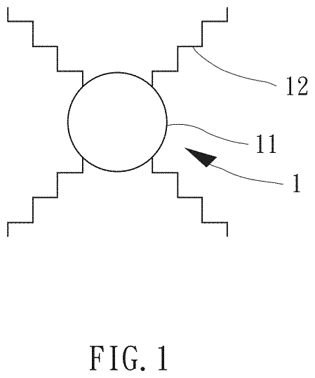

Referring to FIG. 1, a fluorescent probe 1 according to an embodiment of the present invention can include: a silicon oxide (SiOₓ) core 11 and a self-assembled monolayer (SAM) 12.

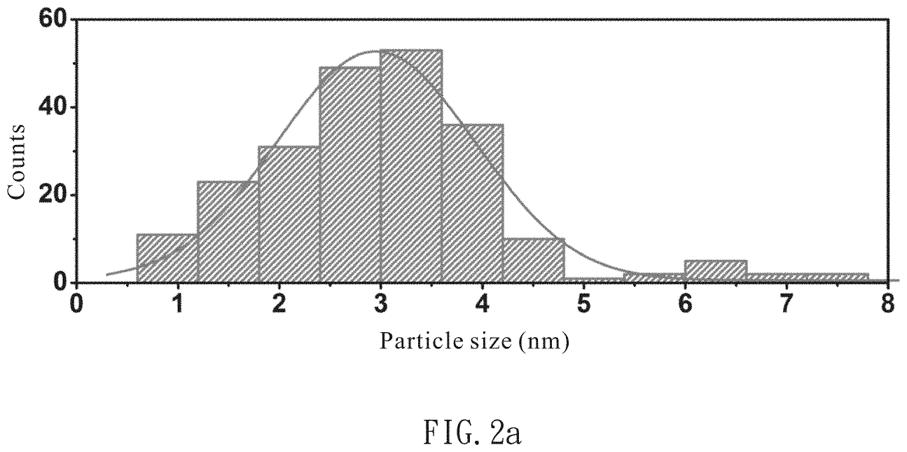

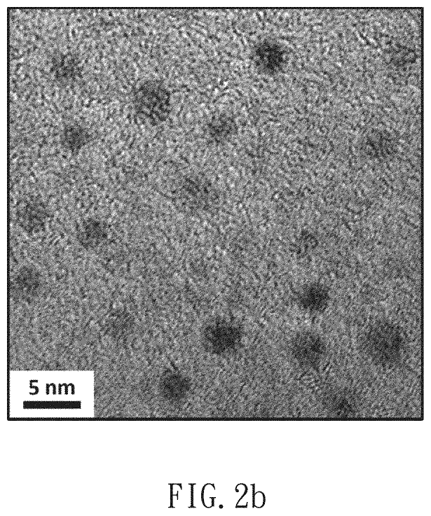

Specifically, 3-glycidoxypropyl trimethoxysilane (GPS) is hydrolyzed and condensed to form the fluorescent probe 1. The self-assembled monolayer 12 can contain an epoxide group, and the self-assembled monolayer 12 joins the silicon oxide core 11 by a covalent bond. In this embodiment, 3-glycidoxypropyl trimethoxysilane (GPS) is hydrolyzed and condensed at 350° C. for 90 minutes under an ambient air atmosphere. After cooling to room temperature, the fluorescent probe 1 is obtained. The fluorescent probe 1 has a pH value about 7. Moreover, with reference to FIGS. 2a and 2b, the fluorescent probe 1 has a particle size of 3.1±0.3 nm.

In addition, the epoxide group of the fluorescent probe 1 can form a conjugated bond with a molecule with an amino group via an aminolysis reaction. Thus, the molecule can be stably immobilized on the self-assembled monolayer 12 of the fluorescent probe 1, forming a nanoparticle including the molecule and the fluorescent probe 1. As an example, the molecule can be various amino acids and proteins with the amino group, or can even be any drugs with the amino group.

It is worthy to be noted that in the situation that the molecule is glucose oxidase, due to the fluorescent probe 1 has the pH value about 7, glucose oxidase immobilized on the fluorescent probe 1 can still show enzymatic activity (glucose oxidase shows enzymatic activity only in the environment with pH value of 4-7.5). Moreover, no EDC/NHS activator is needed when glucose oxidase is immobilized on the fluorescent probe 1, thereby preventing glucose oxidase from depletion due to the complex manufacturing process. In this embodiment, glucose oxidase (2 mg) is dissolved in a phosphate buffer solution (10 μL), followed by being mixed with the fluorescent probe 1 (590 μL). The obtained mixture is sonicated for 30 minutes to form the nanoparticle. All the process is performed under 20-30° C.

The obtained fluorescent probe 1 has a maximum fluorescence emission (λₑₘ) of 503 nm at excitation wavelength (λₑₓ) of 400 nm. The maximum fluorescence emission (λₑₘ) of the fluorescent probe 1 changes when the molecule is immobilized on the fluorescent probe 1. Thus, the maximum fluorescence emission (λₑₘ) can be used to determine whether the molecule able to be immobilized on the fluorescent probe 1 is present in a solution.

Moreover, in the situation that the molecule is glucose oxidase, the fluorescent probe 1 on which glucose oxidase is immobilized is able to be applied to detection of glucose concentration in a glucose solution. For easily understanding, the fluorescent probe 1 on which glucose oxidase is immobilized is named as GOx-GPS-SAND.

In the use of detecting glucose concentration, the GOx-GPS-SAND is added in the glucose solution. The glucose molecule in the glucose solution is oxidized by glucose oxidase, and hydrogen peroxide (H₂O₂) molecule is therefore formed. Then H₂O₂ molecule fluorescent quenches the fluorescent probe 1, resulting in a decrease in fluorescent intensity of the fluorescent probe 1. That is, a worker can calculate the glucose concentration of the glucose solution via the decrease in fluorescent intensity of the fluorescent probe 1.

To validate that the maximum fluorescence emission (λₑₘ) of the fluorescent probe 1 changes when the molecule is immobilized on the fluorescent probe 1, as well as that the GOx-GPS-SAND can be applied to detection of glucose concentration, the following trials are carried out.

Trial (A).

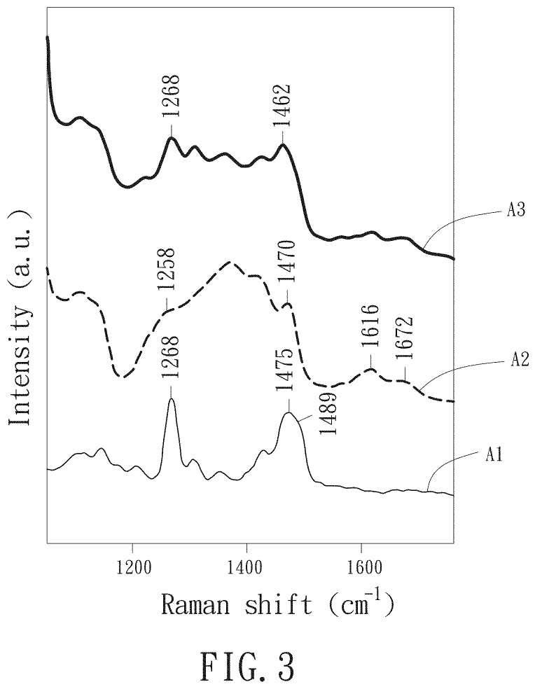

The fluorescent probe 1 is analyzed using SERS (surface-enhanced Raman scattering spectrum), and the SERS spectra are shown in FIG. 3. The peaks at 1268, 1475 and 1489 cm⁻¹ observed in fluorescent probe 1 (group A1) are characteristics of epoxide ring vibrations. The peaks at 1258, 1672 and 1470 cm⁻¹ observed in glucose oxidase (group A2) are assigned to amide III, amide I and CH₂/CH₃ deformation, respectively. However, the peaks at 1268, 1475 and 1489 cm⁻¹ disappear in the GOx-GPS-SAND (group A3).

Trial (B).

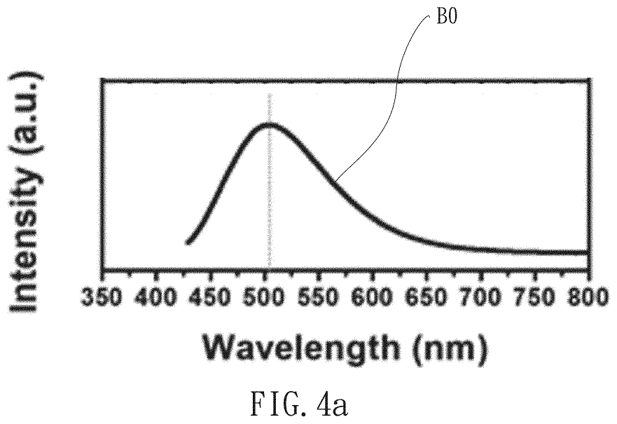

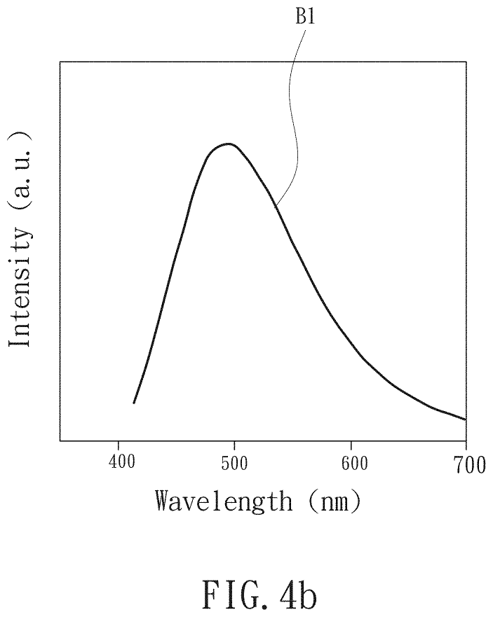

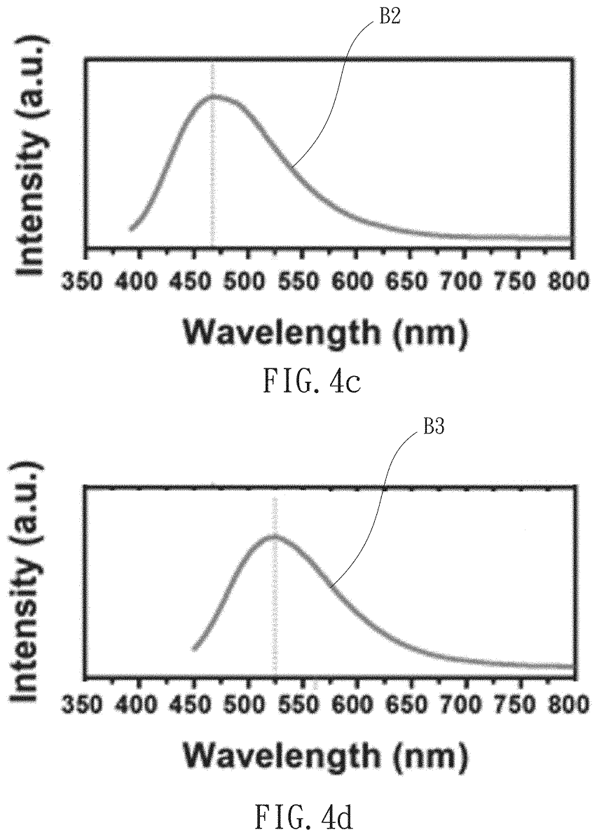

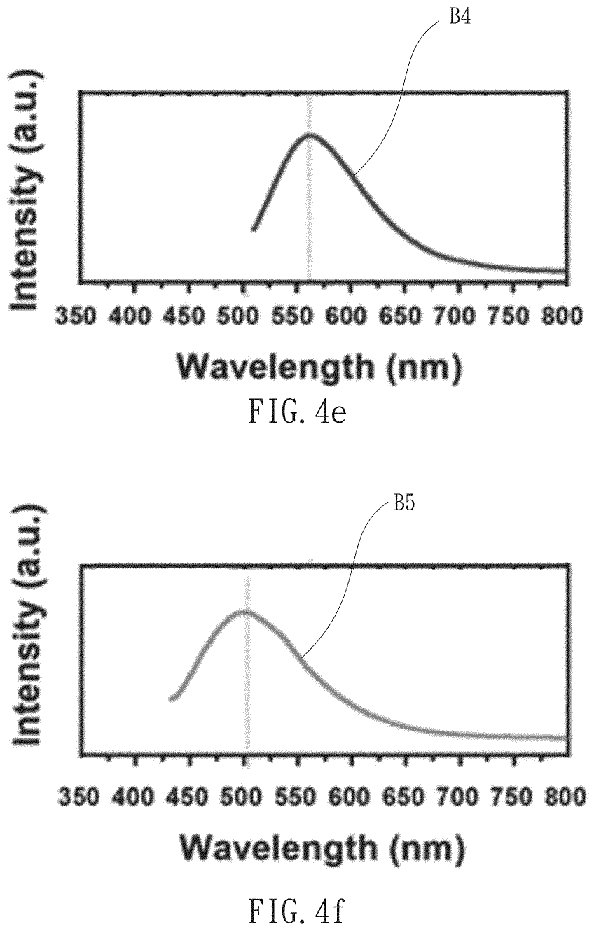

The fluorescent probe 1 (group B0) is analyzed using PL spectrum (photoluminescence spectrum) at the excitation wavelength of 400 nm. FIG. 4a shows the emission spectra recorded from 350 nm to 800 nm, indicating the maximum fluorescence emission (λₑₘ) of the fluorescent probe 1 is 503 nm. That is, the fluorescent probe 1 has the maximum fluorescent intensity at 503 nm.

The fluorescent probe 1 is then added to a solution including the molecule with the amino group, followed by analyzing using PL spectrum at the excitation wavelength of 400 nm. Referring to FIG. 4b, when the solution includes glucose oxidase as the molecule, the maximum fluorescence emission (λₑₘ) of the fluorescent probe 1 is 497 nm (group B1). Referring to FIG. 4c, when the solution includes cisplatin as the molecule, the maximum fluorescence emission (λₑₘ) of the fluorescent probe 1 is 467 nm (group B2). Referring to FIG. 4d, when the solution includes arginine as the molecule, the maximum fluorescence emission (λₑₘ) of the fluorescent probe 1 is 543 nm (group B3). Referring to FIG. 4e, when the solution includes octadecylamine as the molecule, the maximum fluorescence emission (λₑₘ) of the fluorescent probe 1 is 563 nm (group B4). Moreover, the maximum fluorescence emissions (λₑₘ) of the fluorescent probe 1 of groups B1-B4 differ from the maximum fluorescence emission (λₑₘ) of the fluorescent probe 1 of group B0, indicating that the above-mentioned molecule with the amino group can be immobilized on the fluorescent probe 1, respectively.

Besides, the fluorescent probe 1 is added to a solution including octadecane without the amino group, followed by analyzing using PL spectrum at the excitation wavelength of 400 nm. Referring to FIG. 4f, the maximum fluorescence emission (λₑₘ) of the fluorescent probe 1 is still 503 nm (group B5), the same with the maximum fluorescence emission (λₑₘ) of the fluorescent probe 1 of group B0, indicating that octadecane without the amino group cannot be immobilized on the fluorescent probe 1.

Trial (C).

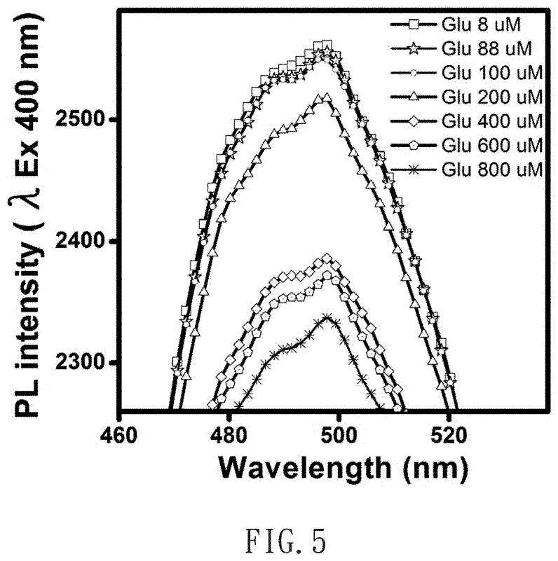

The GOx-GPS-SAND is mixed with 1 μL of glucose at various different concentrations ranging from 8 to 800 μM. The mixture is sonicated for 20 minutes at room temperature, followed by being analyzed by PL spectrum. With reference to FIG. 5, the maximum fluorescent intensity at 497 nm of the fluorescent probe 1 decreases as the glucose concentration increases. Moreover, the decrease in fluorescent intensity at 497 nm is linearly correlated with the glucose concentration of the glucose solution, which varies from 88 μM to 400 μM (R²=0.99).

Accordingly, the fluorescent probe according to an embodiment of the present invention can emit fluorescence, and the epoxide group of the fluorescent probe can form a conjugated bond with a molecule with an amino group (such as various amino acids and proteins) via an aminolysis reaction. Thus, the molecule can be stably immobilized on the self-assembled monolayer of the fluorescent probe. Moreover, the maximum fluorescence emission (λₑₘ) of the fluorescent probe changes due to the immobilization of the molecule. Thus, the maximum fluorescence emission (λₑₘ) of the fluorescent probe can be used to determine whether the molecule able to be immobilized on is present in a solution.

In addition, in the situation that the molecule is glucose oxidase, due to the fluorescent probe has a pH value about 7, glucose oxidase immobilized on the fluorescent probe can still show enzymatic activity.

Moreover, the fluorescent probe on which glucose oxidase is immobilized can also be applied to detection of glucose concentration in the glucose solution. The glucose molecule in the glucose solution is oxidized by glucose oxidase to form the H₂O₂ molecule. Then, the H₂O₂ molecule fluorescent quenching the fluorescent probe, resulting in the change in fluorescent intensity of the fluorescent probe. That is, a worker can calculate the glucose concentration of the glucose solution via the change in fluorescent intensity of the fluorescent probe.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A fluorescent probe obtained via hydrolysis and condensation reaction using 3-glycidoxypropyl trimethoxysilane, comprising:
a silicon oxide core; and
a self-assembled monolayer having an epoxide group, wherein the self-assembled monolayer joins the silicon oxide core by a covalent bond.
